(19) United States
(12) Patent Application Publication
(10) Pub. No.: US 2010/0076487 A1
(43) Pub. Date: Mar. 25, 2010

(54) Kit Containing Combination Absorbable Staple and Non-absorbable Suture, And Method Of Using Same

(76) Inventor: Omer A. Ilahi, Houston, TX (US)

Correspondence Address: Law Offices of Tim Headley, 7941 Katy Fwy, Suite 506, Houston, TX, 77024-1924, US

(21) Appl. No.: 12/236,072

(22) Filed: Sep. 23, 2008

(51) Int. Cl. A61B 17/04

(52) U.S. Cl. 606/232

(57) ABSTRACT

A soft tissue attachment kit, for attaching a patient's soft tissue to a patient's bone at a soft tissue attachment site that is underneath muscle and fat, includes:
a. a collection of suture-staples of various sizes, each suture-staple including:
   i. two spikes, each spike having a base; and
   ii. a suture embedded in each spike, and connecting the bases of the two spikes;
b. a collection of double-barrel insertion cannulas for:
   i. receiving one of the suture-staples; and
   ii. allowing the suture-staple to pass through;
   c. a collection of double-barrel impactors, for tapping the suture-staple into the patient's bone; and
d. a collection of insertion sheaths, each insertion sheath adapted for
   i. penetrating the patient's fat and muscle overlying the soft tissue attachment site;
   ii. receiving the double-barrel insertion cannula; and
   iii. allowing the double-barrel insertion cannula to pass through the insertion sheath to contact the patient's bone.

### CROSS-REFERENCES TO RELATED APPLICATIONS

None.

### STATEMENT REGARDING FEDERALLY SPONSORED RESEARCH OR DEVELOPMENT

None.

### REFERENCE TO A “SEQUENCE LISTING,” A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC

None.

### BACKGROUND OF THE INVENTION

(1) Field of the Invention

The invention relates to securing (fixating) soft tissue to bone in musculoskeletal surgery, and more particularly to fixating soft tissue under tension into a bone tunnel or trough.

(2) Description of the Related Art

Often, the soft tissue needs to be secured under tension. Examples of this include repairing tendons or ligaments that have torn off their normal insertion onto bone. There are several techniques used to accomplish this. One popular method is to create a trough or tunnel in the bone, and then insert the end of the soft tissue structure into it, followed by securing it in place with suture through drill holes in the bone, or by inserting a fixation device. Minimally invasive approaches do not allow for easily creating drill holes and passing sutures through them, and so the use of fixation devices has increased, as a direct result of the increasing popularity of minimally invasive musculoskeletal procedures, such as arthroscopic surgery.

A common fixation device currently used for fixation of soft tissue in a bone tunnel is an interference screw. This is a screw made of metal, plastic, or absorbable material that is inserted into the tunnel, parallel to the soft tissue already inserted, thereby pressing it firmly against the tunnel walls, providing firm fixation. When the soft tissue is under tension, it needs to be kept in place as the fixation device is deployed. This, in turn, is most easily accomplished by drilling a small hole from the apex of the tunnel out the other side of the bone. A suture through the soft tissue can then be passed through the drill hole. Traction on this suture can pull the end of the soft tissue structure into the tunnel and hold it there whilst the definitive fixation device is inserted.

Such interference screws cannot be used to secure soft tissue to a bone trough, however. A popular way of securing soft tissue to a bone trough is with a suture anchor—an anchoring device with attached suture. The anchor portion is inserted into the bone trough with the suture portion passed through the soft tissue to be attached. Tying the suture pulls the soft tissue into the bone trough and secures it there. Some anchors pull the suture through a tortured path or through a loop of itself in such a way that the suture loop can be closed without any formal knot tying.

Other devices are tacks or staples that are either passed through the soft tissue (always the case with a tack, and sometimes the case with a staple) or around the soft tissue (sometimes the case with a staple, never with a tack) and into bone to provide fixation of the soft tissue.

Interference screws, suture anchors, or tacks can be made of metal, plastic, absorbable polymers, or bone from humans (allograft) or even animals (xenograft). Staples used for fixation of soft tissue to bone are typically metallic.

U.S. Pat. No. 6,544,281 (“the '281 patent”) discloses a method for securing soft tissue to bone, by the steps of forming a hole in a bone at a location at which a soft tissue graft is to be affixed; attaching at least one suture to the soft tissue graft; preloading a cannulated tissue anchor on a tissue anchor driver; capturing the suture attached to the graft at a distal end of the driver and inserting the suture into the hole using the driver; drawing the soft tissue graft to the hole; and installing the cannulated tissue anchor into the hole to secure the suture in the hole by interference-friction fixation between the tissue anchor and the bone forming the hole (Claim 1). The entire disclosure of U.S. Pat. No. 6,544,281 is incorporated herein by this reference.

Sometimes, however, there are important structures on the side opposite the blind bone tunnel. In that case, drilling out the other side to create a small passage through which a traction suture may be passed risks injuring the structures on the other side of the bone. Apparently the only commercial embodiment of the device of the '281 patent is the “Bio-tenodesis Screw”, sold by Arthrex, Inc. in Naples, Fla. In that system the end of the soft tissue is secured to the tip of a special screwdriver with suture, brought through the cannulated shaft of the screwdriver. The tip of the screwdriver is then inserted into the already created bone tunnel, bringing the end of the soft tissue with it. An absorbable, cannulated screw is then advanced over the screwdriver to secure the tendon in place. The screwdriver is then removed by releasing the suture attaching it to the soft tissue. Problems with this device include suture breakage as the screw driver is turned many times during screw advancement, damage to the soft tissue by the advancing screw, and wrapping of the soft tissue around the screw as it is being advanced, leading to changing position of the soft tissue in the bone tunnel. Additionally, the presence of a large screw-type device can be problematic, even if made of absorbable material. Large absorbable devices often don't get fully reabsorbed, leading to the formation of localized foreign body reactions that can weaken bones, resulting in fracture, or affect future surgical intervention should the need arise. On the other hand, metal screws can become dislodged over time and lead to irritation of surrounding soft tissues.

U.S. Pat. No. 6,645,227 (“the '227 patent”) discloses a suture anchor. The entire disclosure of U.S. Pat. No. 6,645,227 is incorporated herein by this reference. Suture anchors avoid some of these problems because they tend to be much smaller than interference screws. But suture management can be an issue and tangled suture can complicate the procedure.

U.S. Published Patent Application No. 20070162030 discloses an absorbable tack. The entire disclosure of U.S. Published Patent Application No. 20070162030 is incorporated herein by this reference. Absorbable tacks have had a poor record of maintaining fixation of soft tissue to bone long enough to allow firm healing to occur. There is a natural weak point where the broad head of a tack meets the narrow shaft, and consequently failure at this juncture often occurs, compromising surgical results.

U.S. Pat. No. 5,209,756 (“the '227 patent”) discloses a ligament fixation staple. The entire disclosure of U.S. Pat. No. 5,209,756 is incorporated herein by this reference. Staples are often metallic. Removing metal staples can be problematic; and imaging the adjacent area with subsequent magnetic resonance imaging or computed tomography is compromised by the metal of the staple.

U.S. Published Patent Application No. 20050149033 discloses a bioabsorbable staple. The entire disclosure of U.S. Published Patent Application No. 20050149033 is incorporated herein by this reference. Unfortunately, a common problem with large bioabsorbable stapes and tacks is a large mass of indefinitely partially degraded absorbable material. That is, often some of it is never absorbed.

In light of the foregoing, a need remains for a suture-staple that has the advantages of both absorbable and non-absorbable tacks, screws, and staples, and that will not damage the soft tissue during insertion of the soft tissue into the bone.

### BRIEF SUMMARY OF THE INVENTION

A soft tissue attachment kit, for attaching a patient's soft tissue to a patient's bone, comprises:
a. a collection of suture-staples of various sizes adapted for attaching the patient's soft tissue to the patient's bone, each suture-staple comprising:
   i. two spikes separated by a span ranging from four millimeters to sixteen millimeters; wherein each spike has a base, each spike has the same diameter, the diameter ranging from two millimeters to five millimeters; and each spike has the same total length, the total length ranging from four millimeters to twenty millimeters; and
   ii. a suture firmly embedded in each spike, and connecting the bases of the two spikes, the suture ranging in size from number zero to number five;
b. a collection of double-barrel insertion cannulas, each cannula adapted for:
   i. contacting the patient's bone with individually cannulated feet with serrated tips;
   ii. receiving one of the suture-staples; and
   iii. allowing the suture-staple to pass through to the patient's bone;
c. a collection of double-barrel impactors, each impactor adapted for tapping the suture-staple into the patient's bone; and
d. a collection of insertion sheaths, each insertion sheath adapted for
   i. penetrating the patient's fat and muscle overlying the soft tissue attachment site;
   ii. receiving the double-barrel insertion cannula; and
   iii. allowing the double-barrel insertion cannula to pass through the insertion sheath to contact the patient's bone.

In another feature of the invention, the kit further comprises a right-angle positioning cannula adapted for tensioning a soft tissue into position, prior to attaching the patient's soft tissue to the patient's bone.

A method for attaching, underneath a patient's muscle and fat, the patient's selected soft tissue to the patient's selected bone at a first attachment site, comprises the steps of:
a. inserting an insertion sheath through the patient's muscle and fat to the patient's selected soft tissue;
b. inserting a double-barrel insertion cannula having serrated feet through the insertion sheath;
c. removing the insertion sheath;
d. contacting the patient's selected soft tissue with the serrated feet;
e. drilling, through the double-barrel insertion cannula, two holes in the patient's selected bone at the first attachment site;
f. inserting a suture-staple, having two spikes, through the double-barrel insertion cannula;
g. contacting the suture-staple with a double-barrel impactor until the spikes are seated in the two holes; and
h. removing the double-barrel impactor and the double-barrel insertion cannula.

In another feature of the invention, the method comprises repeating the steps with a second suture-staple at a second attachment site.

### BRIEF DESCRIPTION OF THE SEVERAL VIEWS OF THE DRAWINGS

FIG. 1(a) and FIG. 1(b) are top and side views of the suture-staple of the present invention.

FIG. 2(a) and FIG. 2(b) are top and side views of a double-barrel insertion cannula for the suture-staple of FIG. 1.

FIG. 3(a) and FIG. 3(b) are top and side views of an impactor used to insert the suture-staple of FIG. 1 through the insertion cannula of FIG. 2.

FIG. 4(a) and FIG. 4(b) are top and side views of an insertion sheath used with the insertion cannula of FIG. 2.

FIG. 5(a) and FIG. 5(b) are top and side views of a right angle positioning cannula.

### DETAILED DESCRIPTION OF THE INVENTION

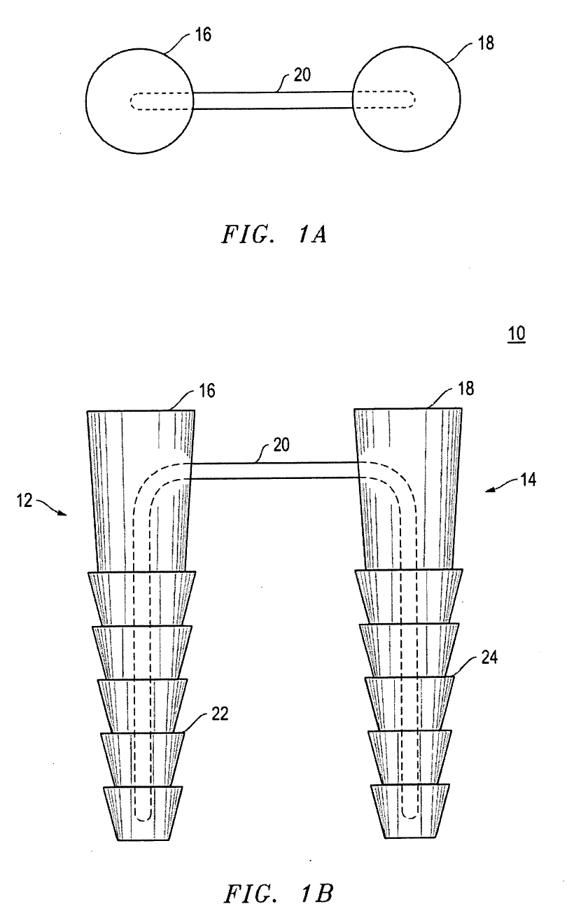

In FIG. 1, the suture-staple 10 of the present invention is shown. The suture-staple 10 includes two spikes 12, 14 with their bases 16, 18 connected with a suture 20. The suture 20 is firmly embedded in each spike 12, 14, and connects the bases 16, 18. The two spikes 12, 14 are separated by a span ranging from four millimeters to sixteen millimeters. The diameter of each base 16, 18 is the same, ranging from two millimeters to five millimeters. The total length of each spike 12, 14 is the same, ranging from four millimeters to twenty millimeters.

In the preferred embodiment, the suture 20 is a size number 2, non-absorbable, and braided, but could also be absorbable or non-braided, and can range in size from number zero to number five. The soft tissue can be a ligament, a tendon, or any other soft connective tissue. In the preferred embodiment the spikes 12, 14 have barbs 22, 24, but may also have smooth or rough walls. The spikes 12, 14 are made of absorbable material, but could also be made of non-absorbable plastic, metal, or even bone (as, for example, allograft or xenograft).

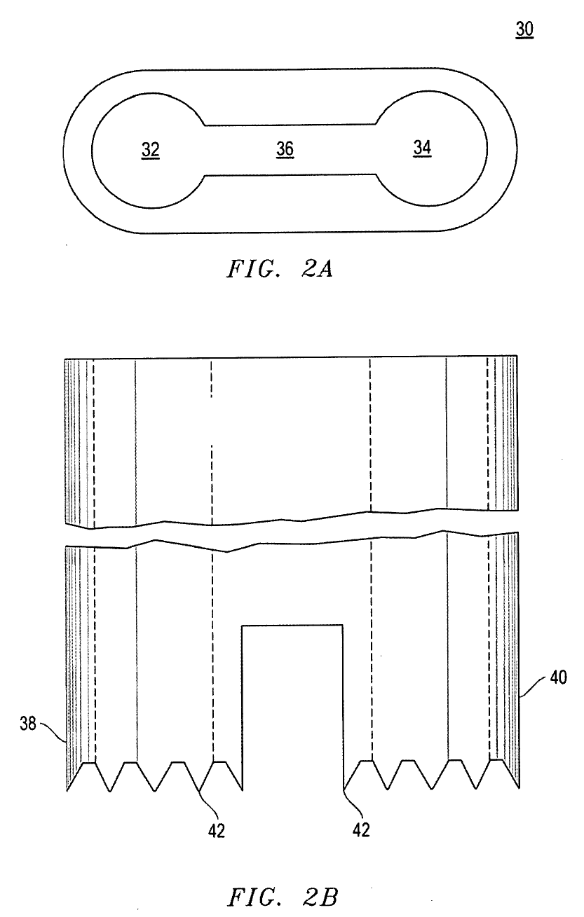

Referring now to FIG. 2, a double-barrel insertion cannula 30 is used to insert the spikes 12, 14. The double-barrel insertion cannula 30 is metal, and includes two parallel cannulas 32, 34 connected to each other with a small channel 36 large enough to let the suture 20 pass. The feet 38, 40 of each cannula 32, 34 are serrated, having teeth 42 to help avoid migration of the double-barrel cannula 30 during the insertion process.

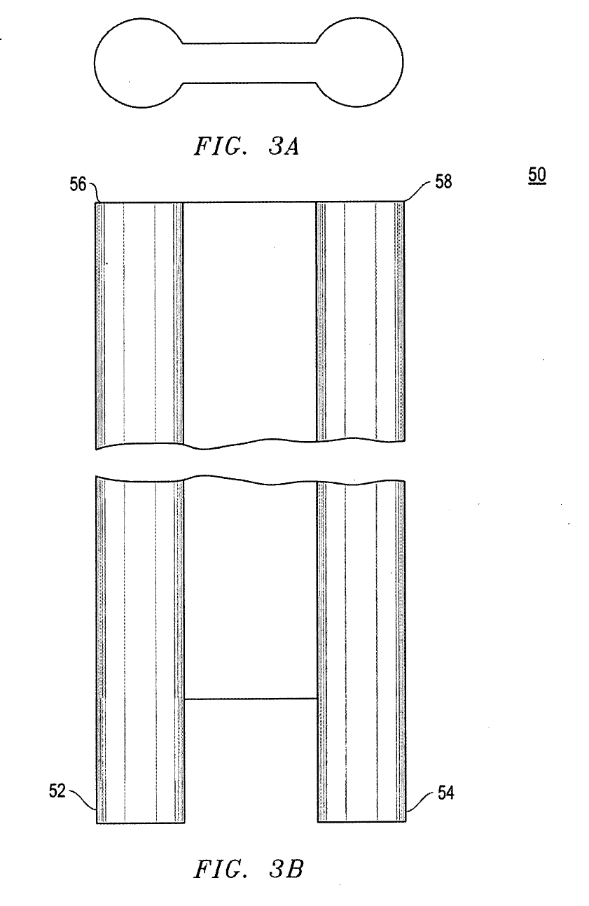

Referring now to FIG. 3, a double-barrel impactor 50 is metal, and is used to tap the suture-staple 10 through the double-barrel insertion cannula 30 into the patient's bone. In operation, the feet 52, 54 of the impactor 50 make contact with the bases 16, 18 of the suture-staple 10, and appropriate force is applied to the tops 56, 58 of the impactor 50.

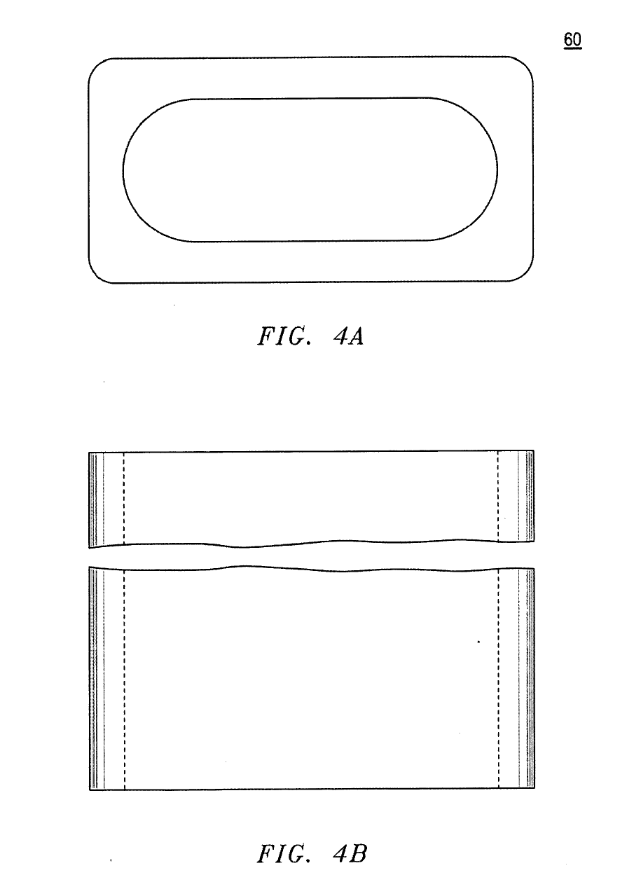

Referring now to FIG. 4, an insertion sheath 60 is used to help insert the double-barrel insertion cannula 30 through layers of muscle and fat, as is typically the case in arthroscopic procedures. The sheath 60 is removed once the serrated feet 38, 40 of the double-barrel insertion cannula 30 have been placed as desired on the soft tissue to be affixed, or on bone adjacent to the soft tissue, as the case requires.

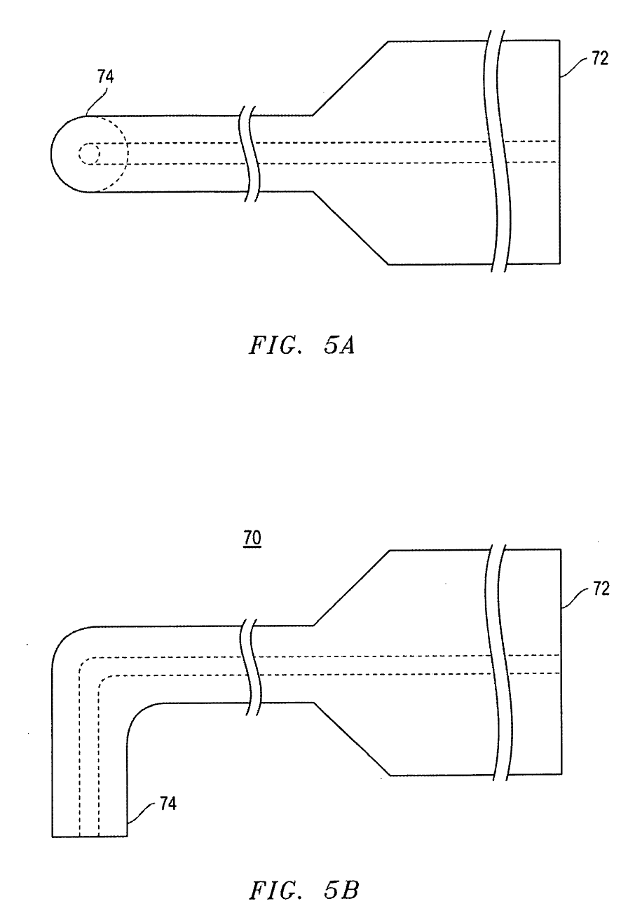

Often, the surgery requires that the soft tissue be held in place, under tension, while the double-barrel insertion cannula 30 is being positioned. Referring now to FIG. 5, in order to both tension the soft tissue, and to hold the soft tissue in place at the attachment site, traction sutures through the end of soft tissue to be fixed to a bone are passed through a right-angle positioning cannula 70. Pulling on these traction sutures, when exiting a handle 72 of the right-angle positioning cannula 70, pulls the end of the soft tissue to the tip 74 of the right-angle positioning cannula 70. The tip 74 is directed to the depth of a bone tunnel or trough at the attachment location.

Referring again to FIGS. 1-5, the ligament attachment kit of the present invention comprises a collection of suture-staples 10 of various sizes, having sutures 20 of various sizes, double-barrel insertion cannulas 30 of various sizes, double-barrel impactors 50 of various sizes, insertion sheaths 60 of various sizes, and the right-angle positioning cannula 70.

According to the method of the present invention, the preferred method to attach a patient's soft tissue to the patient's bone at a first attachment site comprises the following steps:
a. inserting the insertion sheath 60 through the patient's muscle and fat to the patient's selected soft tissue;
b. inserting the double-barrel insertion cannula 30 through the insertion sheath 60;
c. removing the insertion sheath 60;
d. contacting the patient's selected soft tissue with the teeth 42 of the feet 38, 40;
e. drilling, through the double-barrel insertion cannula 30, two holes in the patient's selected bone at the first attachment site;
f. inserting a first suture-staple 10, having two spikes 12, 14, through the double-barrel insertion cannula 30;
g. contacting the suture-staple 10 with the double-barrel impactor 50 until the spikes 12, 14 are seated in the two holes; and
h. removing the double-barrel impactor 50 and the double-barrel insertion cannula 30.
   This process can be repeated as needed with a second suture-staple 10 at a second attachment site.

According to another feature of the method of the present invention, the method of attaching a soft tissue to a bone also includes the following steps:
a. directing the tip 74 of the right-angle positioning cannula 70 to the depth of a bone tunnel or trough;
b. attaching a traction suture to the soft tissue;
c. passing the traction suture into the tip 74 and out through the handle 72;
d. pulling on the traction suture.

1. A soft tissue attachment kit, for attaching a patient's soft tissue to a patient's bone at a soft tissue attachment site that is underneath muscle and fat, the kit comprising:
a. a collection of suture-staples of various sizes adapted for attaching the patient's soft tissue to the patient's bone, each suture-staple comprising:
i. two spikes separated by a span ranging from four millimeters to sixteen millimeters; wherein each spike has a base, each spike has the same diameter, the diameter ranging from two millimeters to five millimeters; and each spike has the same total length, the total length ranging from four millimeters to twenty millimeters; and
ii. a suture embedded in each spike, and connecting the bases of the two spikes, the suture ranging in size from number zero to number five;
b. a collection of double-barrel insertion cannulas, each cannula adapted for:
i. contacting the patient's bone with individually cannulated feet with serrated tips;
ii. receiving one of the suture-staples; and
iii. allowing the suture-staple to pass through to the patient's bone;
c. a collection of double-barrel impactors, each impactor adapted for tapping the suture-staple into the patient's bone; and
d. a collection of insertion sheaths, each insertion sheath adapted for
i. penetrating the patient's fat and muscle overlying the soft tissue attachment site;
ii. receiving the double-barrel insertion cannula; and
iii. allowing the double-barrel insertion cannula to pass through the insertion sheath to contact the patient's bone.

2. The kit of claim 1, wherein the kit further comprises a right-angle positioning cannula adapted for tensioning the soft tissue into position, prior to attaching the soft tissue to the patient's bone.

3. The kit of claim 1, wherein the total length is greater than the span, and the span is greater than the diameter.

4. The kit of claim 1, wherein the total length is substantially equal to the span, and the span is greater than the diameter.

5. The kit of claim 1, wherein the total length is less than the span, and the span is greater than the diameter.

6. The kit of claim 1, wherein the spans range from four millimeters to sixteen millimeters.

7. The kit of claim 6, wherein the diameters range from two millimeters to five millimeters.

8. The kit of claim 7, wherein the total length ranges from four millimeters to twenty millimeters.

9. The kit of claim 8, wherein the sutures range in size from number zero to number five.

10. A method for attaching, underneath a patient's muscle and fat, a selected soft tissue to a selected bone at a first attachment site, comprising the steps of:
a. inserting an insertion sheath through the patient's muscle and fat to the selected soft tissue;
b. inserting a double-barrel insertion cannula having serrated feet through the insertion sheath;
c. removing the insertion sheath;
d. contacting the selected soft tissue with the serrated feet;
e. drilling, through the double-barrel insertion cannula, two holes in the selected bone at the first attachment site;
f. inserting a first suture-staple, having two spikes, through the double-barrel insertion cannula;
g. contacting the first suture-staple with a double-barrel impactor until the spikes are seated in the two holes; and
h. removing the double-barrel impactor and the double-barrel insertion cannula.

11. The method of claim 10, wherein the steps are repeated with the selected soft tissue and the selected bone, using a second suture-staple at a second attachment site.

12. A suture-staple, for attaching a patient's ligament to a patient's bone, comprising:
a. a first spike having
i. a first barbed outer surface;
ii. a first internal cannula for receiving a suture; and
iii. a first base for accepting both continuous and sporadic pressure;
b. a second spike having
i. a second barbed outer surface;
ii. a second internal cannula for receiving the suture; and
iii. a second base for accepting both continuous and sporadic pressure; and
c. a suture embedded in the first and second internal cannula;
wherein the suture is non-absorbable, the spikes are absorbable, each spike has a base of the same diameter, each spike has the same total length, and the spikes are separated by a span, the span being the length of the suture between the spikes.

13. The suture-staple of claim 12, wherein the spikes are non-absorbable.

14. The suture-staple of claim 12, wherein the total length is greater than the span, and the span is greater than the diameter.

15. The suture-staple of claim 12, wherein the total length is substantially equal to the span, and the span is greater than the diameter.

16. The suture-staple of claim 12, wherein the total length is less than the span, and the span is greater than the diameter.
